SEQUENCE LISTING (43 sequences)

SEQ ID NO: 1 - DNA, 56 bp, Artificial Sequence
  aptamer 1
ccgaaaccagaccaccccaccaaggccactcggtcgaaccgccaacactcacccca

SEQ ID NO: 2 - DNA, 56 bp, Artificial Sequence
  aptamer 2
accactgcacgacccccaccagtcccactcgcagcgtccatggcccccacgcccca

SEQ ID NO: 3 - DNA, 56 bp, Artificial Sequence
  aptamer 3
cgcccccacaccaccgccacgaccccacaatcccccgaggtcccccgcgtccacac

SEQ ID NO: 4 - DNA, 56 bp, Artificial Sequence
  aptamer 4
ccagcctcgataccatacccaccaacccaaccagactccacacacccacgcgtctc

SEQ ID NO: 5 - DNA, 56 bp, Artificial Sequence
  aptamer 5
caagcgctccatccaccgacatacctaccaaacactctccttgcccataaaaccac

SEQ ID NO: 6 - DNA, 54 bp, Artificial Sequence
  aptamer 6
cccgccattcccctacataacacctacccatctcccttcccagttaatcaccgc

SEQ ID NO: 7 - DNA, 56 bp, Artificial Sequnce
  aptamer 7
ccacactgcactaaaccagcgtcccggaccatcacaacctctgcccactagcccct

SEQ ID NO: 8 - DNA, 55 bp, Artificial Sequence
  aptamer 8
aactagcccgagccacaatcccataacaagcgtgaccacactatcctgtcttccc

SEQ ID NO: 9 - DNA, 56 bp, Artificial Sequence
  aptamer 9
taactcactccatactcacttgctgattcgccaacaacacacccttaaacagtccc

SEQ ID NO: 10 - DNA, 56 bp, Artificial Sequence
  aptamer 10
ataaccccgacgtacacgccaactatgcccacaacccgccataacccaccaccttc

SEQ ID NO: 11 - DNA, 54 bp, Artificial Sequence
  aptamer 11
cccaagcacaatagccacacccacgacccaccctcatattccgaccacgctccc

SEQ ID NO: 12 - DNA, 56 bp, Artificial Sequence
  aptamer 12
tcccgagcaacaacaactgctcttaaacccccaccaaacacacccggtagaccagc

SEQ ID NO: 13 - DNA, 56 bp, Artificial Sequence
  aptamer 13
cctcaacacacctctaaccaaccctcagcccagcacaacaccccccaaaccgacac

SEQ ID NO: 14 - DNA, 56 bp, Artificial Sequence
  aptamer 14
ctgaataccaacgtaccccctcccaagtccccctacccacgctaaactcaacctca

SEQ ID NO: 15 - DNA, 56 bp, Artificial Sequence
  aptamer 15
tacagccccccaacccaccaccgccgtagataaccacccaccaacgatatcccact

SEQ ID NO: 16 - DNA, 58 bp, Artificial Sequence
  aptamer 16
gccatccgtccccggaacactcacacaccccatccgcaaccccccccactccaccgcc

SEQ ID NO: 17 - DNA, 57 bp, Artificial Sequence
  aptamer 17
gcgcacatattacttcctcaatcaacgcccaccgaacactcccgtcacactacaacc

SEQ ID NO: 18 - DNA, 57 bp, Artificial Sequence
  aptamer 18
ggaccgtttcactcattaccccccatcacacgccacagatactaccccatacaccca

SEQ ID NO: 19 - DNA, 57 bp, Artificial Sequence
  aptamer 19
gtacatacaccgaccaccatcacaagcaccaactcaccaaacatgaactacaccaac

SEQ ID NO: 20 - DNA, 57 bp, Artificial Sequence
  aptamer 20
gtccccatttccagccccttctcattcaccactcacacaaccaatacaaccagccca

SEQ ID NO: 21 - DNA, 57 bp, Artificial Sequence
  aptamer 21
ggtgcgtacccaccccccaaacacccaactcccaccacctcgccaacccgaaaacac

SEQ ID NO: 22 - DNA, 57 bp, Artificial Sequence
  aptamer 22
gcgtgacacctatctaaccaacagccacccatccaacacccgctaaccccactctcg

SEQ ID NO: 23 - DNA, 57 bp, Artificial Sequence
  aptamer 23
gccaatcgccgcacccacccaacccctgccacggctagcaactgcatcatcgcaacc

SEQ ID NO: 24 - DNA, 57 bp, Artificial Sequence
  aptamer 24
gtacctgccctccccgcgttaaaatcacactacaacacaccaatcgtagaaaactaa

SEQ ID NO: 25 - DNA, 62 bp, Artificial Sequence
  aptamer 25
ggccaagcaggtaagtgcggggtccggttggttgttcgggtctcgcgtgcaatatcacgtgt

SEQ ID NO: 26 - DNA, 64 bp, Artificial Sequence
  aptamer 26
ggacaagcatggtgaggctaggttcggcgggtgcggatggcattcggtgggatctttggcgggt

SEQ ID NO: 27 - DNA, 63 bp, Artificial Sequence
  aptamer 27
ggacaagcagaagcggtgagtcggtttgtgtggcatgcggcggtggttgcctgtgtccatcga

SEQ ID NO: 28 - DNA, 65 bp, Artificial Sequence
  aptamer 28
ggccaagcatcgatgcccgtgttggcctgtgcgggggattgtagtgtgcctcgggtgtgcatgag

SEQ ID NO: 29 - DNA, 64 bp, Artificial Sequence
  aptamer 29
ggacaagctcttgtggcggttggccccttagcggttcgggagtttcacagtcacggtcggggtg

SEQ ID NO: 30 - DNA, 66 bp, Artificial Sequence
  aptamer 30
gggcaagctggtataagtatgcaatctgcggtgatatcccatcagtgtgtttggctgtgtctggct

SEQ ID NO: 31 - DNA, 62 bp, Artificial Sequence
  aptamer 31
gtgcaagctgatggttcggtagtttcggatgtttgtgtcgttgctcgcgttgtgaatgtgct

SEQ ID NO: 32 - DNA, 65 bp, Artificial Sequence; identical to SEQ ID NO: 28
  aptamer 32

SEQ ID NO: 33 - DNA, 62 bp, Artificial Sequence; identical to SEQ ID NO: 25
  aptamer 33

SEQ ID NO: 34 - DNA, 62 bp, Artificial Sequence
  aptamer 34
gggcaagctggtataagtatgcaatctgcggtgatatcccatcagtgtgtttggctgtggat

SEQ ID NO: 35 - DNA, 61 bp, Artificial Sequence
  aptamer 35
gccaagccagggcggggtcatgtggttgtttgacttgattgtggccgctcagtgcagccga

SEQ ID NO: 36 - DNA, 63 bp, Artificial Sequence; identical to SEQ ID NO: 27
  aptamer 36

SEQ ID NO: 37 - DNA, 64 bp, Artificial Sequence; identical to SEQ ID NO: 29
  aptamer 37

SEQ ID NO: 38 - DNA, 62 bp, Artificial Sequence
  aptamer 38
gcgggacgcgcgggaggatccgggggttgtgcttgggtggccggatgtccggttattgttgt

SEQ ID NO: 39 - DNA, 62 bp, Artificial Sequence
  aptamer 39
ggcaagctgtccctaggcggtgggtagcaagttcgtgggccgcgcagtgtcttggcagttcc

SEQ ID NO: 40 - DNA, 62 bp, Artificial Sequence; identical to SEQ ID NO: 25
  aptamer 40

SEQ ID NO: 41 - DNA, 62 bp, Artificial Sequence; identical to SEQ ID NO: 34
  aptamer 41

SEQ ID NO: 42 - DNA, 21 bp, Artificial Sequence
  forward primer
tagggaattcgtcgacggatc

SEQ ID NO: 43 - DNA, 22 bp, Artificial Sequence
  reverse primer
ctccaggtcgacgcatgcgccg

(19) United States
(12) Patent Application Publication
(10) Pub. No.: US 2009/0023672 A1
(43) Pub. Date: Jan. 22, 2009

(54) AGE-2 APTAMER

(75) Inventors: Hiroyoshi Inoue, Fukuoka (JP); Yuichiro Higashimoto, Fukuoka (JP); Masayoshi Takeuchi, Toyama (JP); Shoichi Yamagashi, Fukuoka (JP)

Correspondence Address: THE WEBB LAW FIRM, P.C., 700 KOPPERS BUILDING, 436 SEVENTH AVENUE, PITTSBURGH, PA, 15219, US

(73) Assignee: KURUME UNIVERSITY, Fukuoka (JP)

(21) Appl. No.: 11/814,904

(22) PCT Filed: Jan. 17, 2006
(86) PCT No.: PCT/JP2006/300922

§ 371 Date: Jul. 26, 2007

(30) Foreign Application Priority Data
Jan. 27, 2005 (JP) 2005-019381

(51) Int. Cl. A61K 31/7088; C07H 21/00; A61P 3/10; G01N 33/48

(52) U.S. Cl. 514/44; 536/23.1; 436/94

(57) ABSTRACT

An AGE-2 aptamer which binds to a glyceraldehyde-derived advanced glycation end product (AGE-2) but not to human serum albumin and comprises at least 35 bases and in which the cytosine content in the bases is at least 35%, or the guanine content in the bases is at least 32%. Since the AGE-2 aptamer can be used for detecting AGE-2, it can be used as a reagent for detection/diagnosis, and an agent for prevention/treatment of AGE-2 involved diseases such as: diabetic complications such as diabetic retinopathy, diabetic nephropathy, and diabetic neuropathy; neurodegenerative diseases such as Alzheimer's disease; and proliferation, metastasis, and invasion of malignant tumors.

### TECHNICAL FIELD

The present invention relates to a glyceraldehyde-derived advanced glycation end product (AGE-2) aptamer.

### BACKGROUND ART

The term “AGEs (advanced glycation end products)” is a collective term for products by non-enzymatic glycation between reducing sugars such as glucose and proteins. AGEs are considered to be accumulated in the central nerve, and the like, due to aging or diabetes, and to cause diabetic complications such as neuropathy, sensory disorder, and nephropathy. Recently, it has been revealed that AGEs are also involved in neurodegenerative diseases such as Alzheimer's disease, and proliferation, metastasis, and invasion of malignant tumors, for example.

AGEs are produced from various sugars including glucose, and autoxidized and degraded products of glucose. In particular, AGE-2, which is a glyceraldehyde-derived AGE (see FIG. 1), is known to have a highly binding capacity to a receptor for AGEs (RAGE), and to be particularly involved in the onset and development of diabetic angiopathic complications such as diabetic retinopathy and diabetic nephropathy via the RAGE (Yamagishi S. et al., Biochem. Biophys. Res. Commun., 2002, vol. 290, pp. 973-978: and Okamoto T. et al., FASEB J., 2002, vol. 16, pp. 1928-1930). However, there has been little development in diagnosis or blocking agents therefor.

Various methods for measuring AGEs have been investigated. AGEs are yellowish-brown and fluorescent. Therefore, most simply, AGEs are measured utilizing their fluorescence. However, the fluorescence method is low in specificity for and sensitivity to AGEs, and is not particularly suitable for biological samples. Various methods including HPLC, GC/MS, LC/MS or the like can be used to quantify AGEs having specific structures. However, these methods require a long time for measurement, and thus are not suitable for analyzing a large number of samples as in diagnosis.

Currently, an immunoassay is mainly performed using an antibody (anti-CML antibody) which recognizes carboxymethyl lysine (CML), which is one of AGEs whose structure has been elucidated. However, the assay is low in sensitivity, and the antibody itself is expensive. Moreover, CML may be produced not by glycation but by peroxidation of lipids in vivo, and be regarded as a marker for oxidative stress, and thus the anti-CML antibody has a problem for employing as an anti-AGEs antibody. Thus, there is now no antibody for recognizing total AGEs.

Recently, it has been revealed that a single-stranded DNA or RNA molecule can assume such a three-dimensional structure as it can serve as an antibody for recognizing and binding to compounds ranging from low-molecular weight substances to proteins (Ellington A. D. and Szostak J. W., Nature, 1990, vol. 346, pp. 818-822; and Tuerk C. and Gold L., Science, 1990, vol. 249, pp. 505-510). Such a molecule is referred to as an “aptamer”. Aptamers can be obtained from random sequences using the screening method named SELEX (Tuerk C. et al., ibid.).

Aptamers have advantages in that they can be mass synthesized in vitro, may have a stronger binding strength than that of antibodies, and can be stabilized. Accordingly, aptamers can be applied to research, detection, and medical care, likely antibodies. Various studies for such medical applications of aptamers have been reported, including: RNA aptamer for HIV-1 reverse transcriptase (Kensch O. et al., J. Biol. Chem., 2000, vol. 275, pp. 18271-18278), RNA aptamer for complement Cs (Biesecker G. et al., Immunopharm., 1999, vol. 42, pp. 219-230), RNA aptamer for preventing CMV infection (Wang J. et al., RNA, 2000, vol. 6, pp. 571-583), RNA aptamer for vascular endothelial growth factor under development as a therapeutic drug for senile macular degeneration (Ruckman J. et al., J. Biol. Chem., 1988, vol. 273, pp. 20556-20567), aptamer for platelet-derived growth factor with amelioration of symptoms through intravenous injection to rat of mesangium proliferative glomerulonephritis model (Floege J. et al., Am. J. Path., 1999, vol. 154, pp. 169-179), and RNA aptamer for normalizing abnormality caused by overexpression of Drosophila B52 protein (Shi H. et al., Proc. Natl. Acad. Sci. USA, 1999, vol. 96, pp. 10033-10038).

### DISCLOSURE OF INVENTION

It is an object of the present invention to provide an aptamer that can specifically bind to AGE-2.

The present invention provides an aptamer that binds to a glyceraldehyde-derived advanced glycation end product (AGE-2) but not to human serum albumin, wherein the aptamer comprises at least 35 bases, and the cytosine content in the bases is at least 35%, or the guanine content in the bases is at least 32%.

In an embodiment, the aptamer is a single-stranded DNA.

In another embodiment, the aptamer comprises at least 50 bases and not greater than 120 bases.

In another embodiment, the cytosine content in the bases is at least 40%.

In another embodiment, the cytosine content in the bases is at least 50%.

In another embodiment, the guanine content in the bases is at least 35%.

In another embodiment, the guanine content in the bases is at least 40%.

In another embodiment, the single-stranded DNA comprises a base sequence according to any one of SEQ ID NOs: 1 to 24 in the Sequence Listing.

In another embodiment, the single-stranded DNA comprises a base sequence according to any one of SEQ ID NOs: 25 to 41 in the Sequence Listing.

Moreover, the present invention provides an AGE-2 detection reagent including the aptamer described above.

Moreover, the present invention provides an AGE-2 detection kit including the AGE-2 detection reagent.

Moreover, the present invention provides a diagnostic reagent for an AGE-2 involved disease, including the aptamer described above.

Moreover, the present invention provides a diagnostic kit for an AGE-2 involved disease, including the reagent.

Moreover, the present invention provides an anti-AGE-2 agent including the aptamer described above.

Moreover, the present invention provides an agent for preventing or treating an AGE-2 involved disease, including the aptamer described above.

In an embodiment, the AGE-2 involved disease is a diabetic complication.

The present invention provides an AGE-2 aptamer that specifically binds to AGE-2. The AGE-2 aptamer of the invention can be used to qualify or quantify AGE-2. Thus, the AGE-2 aptamer can be used as a reagent for clinical test of diseases such as diabetic complications, neurodegenerative diseases, and malignant tumors. Furthermore, since the AGE-2 aptamer has an activity for inhibiting AGE-2, it can be used as an anti-AGE-2 agent.

Moreover, the AGE-2 aptamer of the present invention can be chemically synthesized at low cost. Furthermore, the aptamer can be stabilized by modification. To the aptamer a fluorescent or luminescent domain can be added to improve efficiency for detection.

### BRIEF DESCRIPTION OF DRAWINGS

FIG. 1 is a diagram illustrating the process of formation of AGE-2, which is a glyceraldehyde-derived AGE.

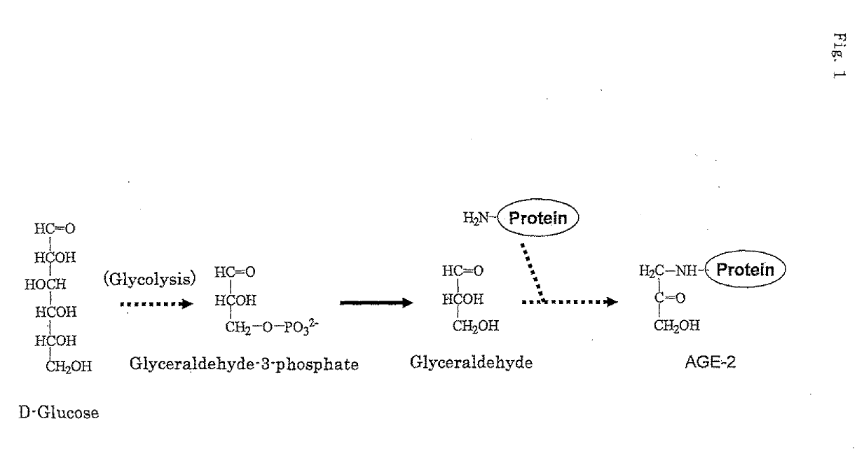

FIG. 2 is a flow chart illustrating the scheme of SELEX process.

FIG. 3 shows fluorescence spectrographies of AGE-2 at varied concentrations (A), and of AGE-2 at 100 μg/mL in combination with an AGE-2 aptamer at varied concentrations (B and C).

FIG. 4 is a graph for illustrating a method for calculating the rate of apoptosis inhibition.

### BEST MODE FOR CARRYING OUT THE INVENTION

As described above, an aptamer is a single-stranded DNA or RNA that can specifically bind to a specific compound. In the present invention, the specific compound is AGE-2. More specifically, the AGE-2 aptamer of the present invention binds to AGE-2, and does not bind to human serum albumin. The AGE-2 aptamer may be either a single-stranded DNA or a single-stranded RNA.

The AGE-2 aptamer of the present invention comprises at least 35 bases, and preferably at least 50 bases and not greater than 120 bases. In the case of 34 bases or less, the aptamer does not bind to AGE-2.

The AGE-2 aptamer of the present invention is preferably rich in either one of cytosine and guanine in the bases constituting the aptamer. If rich in cytosine, then the cytosine content may be at least 35%, at least 40%, or at least 50%. If rich in guanine, then the guanine content in the bases may be at least 32%, at least 35%, or at least 40%. With these base contents, the aptamer more easily binds to AGE-2.

More specifically, a single-stranded DNA comprised of a base sequence according to any one of SEQ ID NOs: 1 to 24 in the Sequence Listing is included in examples of the AGE-2 aptamer of the present invention. This single-stranded DNA is composed of 54 to 58 bases, and has a cytosine content of at least 35% in the bases. A single-stranded DNA comprised of a base sequence according to any one of SEQ ID NOs: 25 to 41 in the Sequence Listing is also included. This single-stranded DNA is composed of 61 to 66 bases, and has a guanine content of at least 32% in the bases.

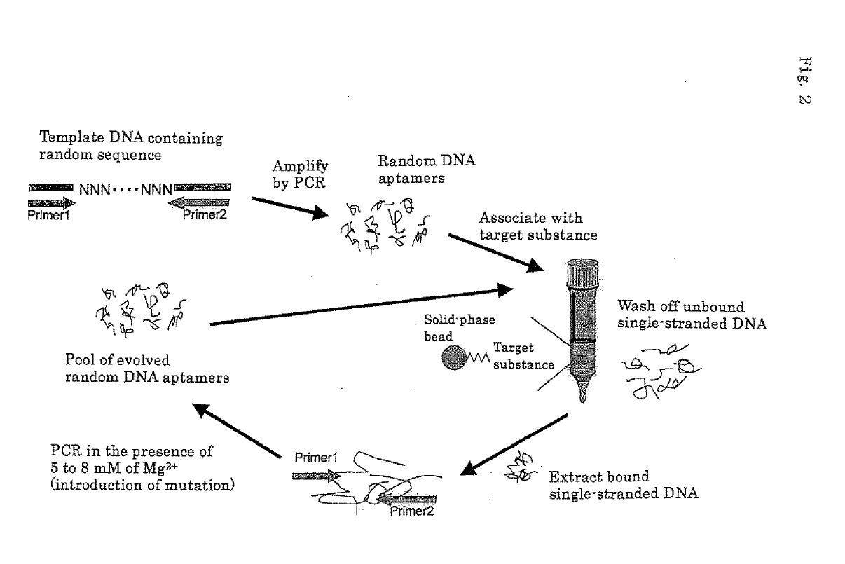

The AGE-2 aptamer of the present invention can be obtained by SELEX (Systematic Evolution of Ligands by EXponential enrichment) method, commonly used for obtaining aptamers. The scheme of the SELEX process using the library of single-stranded DNAs is described with reference to FIG. 2. First, a template DNA is synthesized that contains an appropriate length of random sequence flanked by two arbitrary primer sequences. In the present invention, it is appropriate that the length of the random sequence is 35 bases to 120 bases. This template DNA is amplified by PCR (Polymerase Chain Reaction) to obtain a randomized DNA aptamer pool. Next, the randomized DNA aptamer pool is associated with a target substance, and then DNAs not bound to the target substance are removed, and DNA aptamers bound to the target substance are extracted. The resultant DNA aptamers are amplified by PCR using the primer sequences, wherein the PCR is performed under the presence of 5 to 8 mM of Mg²⁺ for lowering replication accuracy and causing a mutation to be introduced more easily to obtain a further DNA aptamer pool that contains new DNA aptamers that would not be present in the DNA aptamer pool before performing the association with the target substance. The new DNA aptamers may have a stronger binding strength, that is, evolved DNA aptamers may be generated. A series of procedures explained above is repeated for 5 to 15 rounds with a pool of the evolved DNA aptamers to obtain DNA aptamers being able to specifically bind to the target substance. The resultant DNA aptamer pool after the final round is cloned and sequenced as usually performed by those skilled in the art. The procedures such as synthesis of template DNA and PCR in the SELEX process and cloning and sequencing are performed by methods commonly used by those skilled in the art. The AGE-2 aptamer of the present invention can be chemically synthesized by methods commonly used by those skilled in the art based on the determined sequence.

In the present invention, the target substance in the SELEX process is AGE-2, preferably conjugated with human serum albumin (HSA). AGE-2 can be prepared by any methods including incubation method and chemical synthesis method. In the incubation method, for example, human serum albumin (HSA) is incubated with D-glucose for several weeks, or incubated with D-glyceraldehyde or D-glycolaldehyde for several days. The chemical synthesis method is performed, for example, following the method disclosed by Tessier et al. (Biochem. J., 2003, vol. 369, pp. 705-719). More specifically, AGE-2 is prepared by mixing acetyl-lysine and glyceraldehyde in a phosphate buffer solution (pH 7.4), adding diethylenetriaminepentaacetic acid and 25% methanol to the mixture, and incubating the resultant at 37° C. for several days. AGE-2 is preferably immobilized on an appropriate solid phase (e.g., bead etc.) when applied to the SELEX process.

The obtained aptamer can be measured for binding affinity to AGE-2 utilizing an ability of AGE-2 to emit fluorescense as attenuation of the fluorescence intensity of AGE-2 in combination with the aptamer. In this manner, aptamers with a stronger affinity can be further selected from aptamers in the present invention.

Since the AGE-2 aptamer of the present invention specifically binds to AGE-2, it can be used for detecting AGE-2. The AGE-2 aptamer of the present invention can be synthesized using modified nucleotides/nucleotides for the purpose of stabilization. To the AGE-2 aptamer a fluorescent or luminescent domain can be added for the purpose of improving efficiency of detecting the aptamer itself. Thus, the AGE-2 aptamer of the present invention can be used as an AGE-2 detection reagent.

Typically, the AGE-2 aptamer of the present invention can be used in various analyses in which an antibody can be used, such as ELISA and tissue staining, like the antibody. Using a DNA microarray technique, AGE-2 aptamer chips also can be produced. Thus, the AGE-2 aptamer of the present invention can be provided as an AGE-2 detection kit.

Typical examples of a sample which is subject to AGE-2 detection include various biological samples (blood, cell, tissue etc.) and their treated materials. The detection/diagnosis of AGE-2 involved diseases such as diabetic complications such as diabetic retinopathy, diabetic nephropathy, and diabetic neuropathy; neurodegenerative diseases such as Alzheimer's disease; and proliferation, metastasis, and invasion of malignant tumors can be carried out through detecting AGE-2 in the sample. Thus, the AGE-2 aptamer of the present invention can be provided as a reagent for clinical test of or a kit for diagnosis of the above-mentioned diseases.

Furthermore, since the AGE-2 aptamer of the present invention has an activity of inhibiting AGE-2, it can be used as an anti-AGE-2 agent, or as an agent for preventing/treating AGE-2 involved diseases, such as the diseases mentioned above.

Moreover, the AGE-2 aptamer of the present invention also can be used for fundamental researches such as elucidation of the onset mechanism of AGE-2 involved diseases.

### EXAMPLES

### Example 1

### Preparation of AGE-2 Aptamers

(1-1: Preparation of Single-Stranded Random Oligo DNAs)

The single-stranded random oligo DNA containing a random region of 34, 56, or 72 bases and flanking primer sites of SEQ ID NOs: 42 and 43 was synthetically prepared as explained below.

First, a column was filled with CPG (controlled pore glass) carriers to which the 3′ terminal nucleotide was bound via a 3′-hydroxyl group. Next, the protecting group, a dimethoxytrityl group, at position 5′ of ribose was removed using trichloroacetic acid for detritylation. A second nucleotide in which a hydroxyl group at position 3′ of ribose was reacted with cyanoethyl phosphoramidite was coupled to a 5′-hydroxyl group of the detritylated, first nucleotide using a base catalyst (tetrazole), while an unreacted 5′-hydroxyl group was acetylated with acetic anhydride. The linkage between the two nucleotides was oxidized with iodine to convert trivalent phosphorus into pentavalent phosphate ester. The procedure from the detritylation to the conversion into phosphate ester was repeated until the intended chain length. The random region was made using a mixture of four types of nucleotide amidites on the coupling reaction. After the reaction, oligo DNAs were removed from the column by treatment with ammonium, and purified using reverse phase cartridge column, and freeze dried, and dissolved in an appropriate amount of water to provide a template DNA of a library for SELEX.

(1-2: Preparation of AGE-2)

Human serum albumin (HSA) (manufactured by Sigma) was incubated with D-glyceraldehyde under aseptic condition at 37° C. for seven days. Unreacted sugar was removed by dialysis against phosphate buffered saline (PBS). Before use, it was confirmed using an Endospecy ES-20S system (SEIKAGAKU CORPORATION) that there was no endotoxin.

(1-3: Binding of AGE-2 to Beads)

The obtained AGE-2 from the step 1-2 was immobilized on beads, using a SulfoLink (registered trademark) coupling gel (Product Number 20401) manufactured by PIERCE, following the instruction manual of the product as explained below.

First, a column was filled with the coupling gel, and equilibrated with a coupling buffer solution (50 mM Tris-HCl, 5 mM EDTA, pH 8.5). AGE-2 was dissolved in the coupling buffer solution, and the resultant was combined with the coupling gel and incubated at room temperature for one hour. After completion of the reaction, the column was washed for several times with the coupling buffer solution. L-cysteine was dissolved in the coupling buffer solution, and the resultant was combined with the coupling gel and incubated at room temperature for 30 minutes. After completion of the reaction, the column was washed for several times with the coupling buffer solution and PBS. Herein, the degree of immobilization of the AGE-2 was calculated by measuring absorbance before and after the reaction. After the immobilization, the gel (bead) aliquots were stored in a dark cold place before use.

(1-4: Binding of HSA to Beads)

HSA was immobilized on beads, using SulfoLink (registered trademark) coupling gel (Product Number 20401) and UltraLink (registered trademark) EDC/DADPA Immobilization Kit (Product Number 53154) manufactured by PIERCE.

(1-5; SELEX Process)

Using the prepared random oligo DNAs from the step 1-1 as a template, PCR was performed using a forward primer (SEQ ID NO: 42) and a reverse primer (SEQ ID NO: 43) (12 cycles for 94° C., 15 seconds; 55° C., 15 seconds; 72° C., 15 seconds). Following the amplification, a plus strand was amplified by asymmetrical PCR using only the forward primer (45 cycles for 94° C., 15 seconds; 55° C., 15 seconds; 72° C., 15 seconds). The amplified plus strands were purified by agarose gel electrophoresis, and taken for a DNA library for SELEX. The DNA library for SELEX was dissolved in PBS, and the solution was heated at 95° C. for five minutes, and then cooled down to room temperature. Next, the DNA library for SELEX was combined with the AGE-2 immobilized beads from the step 1-3, and incubated at room temperature for 30 minutes. The incubated beads were washed for several times with PBS, and to the washed bead, an appropriate amount of water was added and mixed and the mixture was heated at 100° C. for five minutes to release DNAs from the AGE-2 immobilized beads, and the DNAs were collected. The collected DNAs were combined with the HSA immobilized beads from the step 1-4, and incubated at room temperature for 10 minutes. DNAs that passed through (that did not bind to HSA) were collected and concentrated by ethanol precipitation. Using the concentrated DNAs as a template, a series of procedures explained above was repeated for 5 to 15 rounds, wherein PCR was performed under the presence of 5 to 8 mM of Mg²⁺ for the introduction of mutation.

(1-6: Cloning)

The DNA obtained after the 5 to 15 rounds was amplified by PCR using a forward primer (SEQ ID NO: 42) and a reverse primer (SEQ ID NO: 43), and purified by agarose gel electrophoresis, to obtain an AGE-2 specific DNA. The AGE-2 specific DNA was introduced to a cloning vector (Invitrogen Corporation; Zero Blunt (registered trademark) TOPO (registered trademark) PCR Cloning Kit for Sequencing (Catalog Number K2875J10)), and sequenced in the following manner.

First, the AGE-2 specific DNA (PCR product) was combined with the cloning vector (TOPO vector), and incubated at room temperature for five minutes. After completion of the reaction, a part of the reaction solution was added to competent cells, and incubated under ice-cooling for 30 minutes, and followed by heat-shock at 42° C. for 30 seconds, and the resultant was cooled on ice for two minutes. The cooled reaction solution was added to SOC medium contained in the kit, and incubated at 37° C. for one hour. An appropriate amount of the resultant was plated on an agar plate (LB medium containing 50 μg/mL of ampicillin), and incubated at 37° C. overnight. Several tens of clones were picked up at random, and a plasmid DNA was prepared by alkaline lysis.

(1-7: Sequencing)

Sequencing of the AGE-2 specific DNA in the plasmid DNA from the step 1-6 was performed using an ABI377 manufactured by Applied Biosystems, following BigDye Terminator Cycle sequencing.

Accordingly, no product from the single-stranded DNA containing a random region of 34 bases was bound to AGE-2. AGE-2 aptamers, such as single-stranded DNAs of 54 to 58 bases according to SEQ ID NOs: 1 to 24, were obtained from the single-stranded DNA containing a random region of 56 bases (Table 1). Some products from the single-stranded DNA containing a random region of 72 bases were bound to AGE-2, which were not sequenced.

TABLE 1

| SEQ ID | | Number of Residues | | | | Rate of Contents | | | |
|---|---|---|---|---|---|---|---|---|---|
| NO: | Sequence | A | G | C | T | A | G | C | T |

| | | | | | | | | | |
|---|---|---|---|---|---|---|---|---|---|
| 1 | CCGAAACCAGACCACCCCACCAAGGCCACTCGGTCGAACCGCCAACACTCACCCCA | 17 | 8 | 28 | 3 | 30% | 14% | 50% | 5% |
| 2 | ACCACTGCACGACCCCCACCAGTCCCACTCGCAGCGTCCATGGCCCCCACGCCCCA | 11 | 9 | 31 | 5 | 20% | 16% | 55% | 9% |
| 3 | CGCCCCCACACCACCGCCACGACCCCACAATCCCCCGAGGTCCCCCGCGTCCACAC | 11 | 8 | 34 | 3 | 20% | 14% | 61% | 5% |
| 4 | CCAGCCTCGATACCATACCCACCAACCCAACCAGACTCCACACACCCACGCGTCTC | 16 | 5 | 29 | 6 | 29% | 9% | 52% | 11% |
| 5 | CAAGCGCTCCATCCACCGACATACCTACCAAACACTCTCCTTGCCCATAAAACCAC | 18 | 4 | 25 | 9 | 32% | 7% | 45% | 16% |
| 6 | CCCGCCATTCCCCTACATAACACCTACCCATCTCCCTTCCCAGTTAATCACCGC | 12 | 3 | 27 | 12 | 22% | 6% | 50% | 22% |
| 7 | CCACACTGCACTAAACCAGCGTCCCGGACCATCACAACCTCTGCCCACTAGCCCT | 14 | 7 | 27 | 8 | 25% | 13% | 48% | 14% |
| 8 | AACTAGCCCGAGCCACAATCCCATAACAAGCGTGACCACACTATCCTGTCTTCCC | 16 | 7 | 22 | 10 | 29% | 13% | 40% | 18% |
| 9 | TAACTCACTCCATACTCACTTGCTGATTCGCCAACAACACACCCTTAAACAGTCCC | 17 | 4 | 22 | 13 | 30% | 7% | 39% | 23% |
| 10 | ATAACCCCGACGTACACGCCAACTATGCCCACAACCCGCCATAACCCACCACCTTC | 17 | 5 | 27 | 7 | 30% | 9% | 48% | 13% |
| 11 | CCCAAGCACAATAGCCACACCCACGACCCACCCTCATATTCCGACCACGCTCCC | 15 | 5 | 28 | 6 | 28% | 9% | 52% | 11% |
| 12 | TCCCGAGCAACAACAACTGCTCCTTAAACCCCCACCAAACACACCCGGTAGACCAGC | 19 | 7 | 24 | 6 | 34% | 13% | 43% | 11% |
| 13 | CCTCAACACACCTCTAACCAACCCTCAGCCCAGCACAACACCCCCCAAACCGACAC | 19 | 3 | 30 | 4 | 34% | 5% | 54% | 7% |
| 14 | CTGAATACCAACGTACCCCCTCCCAAGTCCCCCTACCCACGCTAAACTCAACCTCA | 16 | 4 | 27 | 9 | 29% | 7% | 48% | 16% |
| 15 | TACAGCCCCCCAACCCACCACCGCCGTAGATAACCACCCACCAACGATATCCCACT | 17 | 5 | 28 | 6 | 30% | 9% | 50% | 11% |
| 16 | GCCATCCGTCCCCGGAACACTCACACACCCCATCCGCAACCCCCCCCACTCCACCGCC | 12 | 6 | 35 | 5 | 21% | 10% | 60% | 9% |
| 17 | GCGCACATATTACTTCCTCAATCAACGCCCACCGAACACTCCCGTCACACTACAACC | 17 | 5 | 25 | 10 | 30% | 9% | 44% | 18% |
| 18 | GGACCGTTTCACTCATTACCCCCCATCACACGCCACAGATACTACCCCATACACCCA | 16 | 5 | 26 | 10 | 28% | 9% | 46% | 18% |
| 19 | GATACATACACCGACCACCATCACAAGCACCAACTCACCAAACATGAACTACACCAAC | 24 | 4 | 23 | 5 | 43% | 7% | 41% | 9% |
| 20 | GTCCCCATTTCCAGCCCCTTCTCATTCACCACTCACACAACCAATACAACCAGCCCA | 16 | 3 | 27 | 11 | 28% | 5% | 47% | 19% |
| 21 | GGTGCGTACCCACCCCCCAAACACCCAACTCCCACCACCTCGCCAACCCGAAAACAC | 17 | 6 | 30 | 4 | 30% | 11% | 53% | 7% |
| 22 | GCGTGACACCTATCTAACCAACAGCCACCCATCCAACACCCGCTAACCCCACTCTCG | 16 | 6 | 27 | 8 | 28% | 11% | 47% | 14% |
| 23 | GCCAATCGCCGCACCCACCCAACCCCTGCCACGGCTAGCAACTGCATCATCGCAACC | 14 | 9 | 28 | 6 | 25% | 16% | 49% | 11% |
| 24 | GTACCTGCCCTCCCCGCGTTAAAATCACACTACAACACACCAATCGTAGAAAACTAA | 21 | 6 | 20 | 10 | 37% | 11% | 35% | 18% |

As shown in Table 1, all of the obtained AGE-2 aptamers had a cytosine content of 35% or more in the bases constituting the aptamers.

### Example 2

### Preparation of AGE-2 Aptamers

AGE-2 aptamers were prepared as in Example 1, except that a single-stranded random oligo DNA containing a random region of 64 bases and flanking primer sites of SEQ ID NOs: 42 and 43 was used as a template.

As a result, AGE-2 aptamers, such as single-stranded DNAs of 61 to 66 bases according to SEQ ID NOs: 25 to 41, were obtained (Table 2).

TABLE 2

| | | |
|---|---|---|
| | Number | |
| SEQ ID | of Residues | Rate of Contents |

| | | | | | | | | | |
|---|---|---|---|---|---|---|---|---|---|
| NO: | Sequence | A | G | C | T | A | G | C | T |

| | | | | | | | | | |
|---|---|---|---|---|---|---|---|---|---|
| 25 | GGCCAAGCAGGTAAGTGCGGGGTCCGGTTGGTTGTTCGGGTCTCGCGTGCAATATCACGTGT | 9 | 24 | 13 | 16 | 15% | 39% | 21% | 26% |
| 26 | GGACAAGCATGGTGAGGCTAGGTTCGGCGGGTGCGGATGGCATTCGGTGGGATCTTTGGCGGGT | 9 | 30 | 10 | 15 | 14% | 47% | 16% | 23% |
| 27 | GGACAAGCAGAAGCGGTGAGTCGGTTTGTGTGGCATGCGGCGGTGGTTGCCTGTGTCCATCGA | 10 | 26 | 12 | 15 | 16% | 41% | 19% | 24% |
| 28 | GGCCAAGCATCGATGCCCGTGTTGGCCTGTGCGGGGGATTGTAGTGTGCCTCGGGTGTGCATCAG | 8 | 27 | 14 | 16 | 12% | 42% | 22% | 25% |
| 29 | GGACAAGCTCTTGTGGCGGTTGGCCCCTTAGCGGTTCGGGAGTTTCACAGTCACGGTCGGGGTG | 8 | 25 | 15 | 16 | 13% | 39% | 23% | 25% |
| 30 | GGGCAAGCTGGTATAAGTATGCAATCTGCGGTGATATCCCATCAGTGTGTTTGGCTGTGTCTGGCT | 12 | 21 | 12 | 21 | 18% | 32% | 18% | 32% |
| 31 | GTGCAAGCTGATGGTTCGGTAGTTTCGGATGTTTGTGTCGTTGCTCGCGTTGTGAATGTGCT | 7 | 22 | 9 | 24 | 11% | 35% | 15% | 39% |
| 32 | GGCCAAGCATCGATGCCCGTGTTGGCCTGTGCGGGGGATTGTAGTGTGCCTCGGGTGTGCATGAG | 8 | 27 | 14 | 16 | 12% | 42% | 22% | 25% |
| 33 | GGCCAAGCAGGTAAGTGCGGGGTCCGGTTGGTTGTTCGGGTCTCGCGTGCAATATCACGTGT | 9 | 24 | 13 | 16 | 15% | 39% | 21% | 26% |
| 34 | GGGCAAGCTGGTATAAGTATGCAATCTGCGGTGATATCCCATCAGTGTGTTTGGCTGTGGAT | 13 | 20 | 10 | 19 | 21% | 32% | 16% | 31% |
| 35 | GCCAAGCCAGGGCGGGGTCATGTGGTTGTTTGACTTGATTGTGGCCGCTCAGTGCAGCCGA | 9 | 23 | 14 | 15 | 15% | 38% | 23% | 25% |
| 36 | GGACAAGCAGAAGCGGTGAGTCGGTTTGTGTGGCATGCGGCGGTGGTTGCCTGTGTCCATCGA | 10 | 26 | 12 | 15 | 26% | 41% | 19% | 24% |
| 37 | GGACAAGCTCTTGTGGCGGTTGGCCCCTTAGCGGTTCGGGAGTTTCACAGTCACGGTCGGGGTG | 8 | 25 | 15 | 16 | 13% | 39% | 23% | 25% |
| 38 | GCGGGACGCGCGGGAGGATCCGGGGGTTGTGCTTGGGTGGCCGGATGTCCGGTTATTGTTGT | 5 | 30 | 11 | 16 | 8% | 48% | 18% | 26% |
| 39 | GGCAAGCTGTCCCTAGGCGGTGGGTAGCAAGTTCGTGGGCCGCGCAGTGTCTTGGCAGTTCC | 8 | 24 | 16 | 14 | 13% | 39% | 26% | 23% |
| 40 | GGCCAAGCAGGTAAGTGCGGGGTCCGGTTGGTTGGTTCGGGTCTCGCGTGCAATATCACGTGT | 9 | 24 | 13 | 16 | 15% | 39% | 21% | 26% |
| 41 | GGGCAAGCTGGTATAAGTATGCAATCTGCGGTGATATCCCATCAGTGTGTTTGGCTGTGGAT | 13 | 20 | 10 | 19 | 21% | 32% | 16% | 31% |

As shown in Table 2, all of the obtained AGE-2 aptamers had a guanine content of 32% or more in the bases constituting the aptamers.

Then, each AGE-2 aptamer was chemically synthesized according to phosphoamidite method as in the step 1-1, based on the sequence of the obtained AGE-2 aptamer.

### Example 3

### Experiment for AGE-2 Fluorescence Inhibition by AGE-2 Aptamers

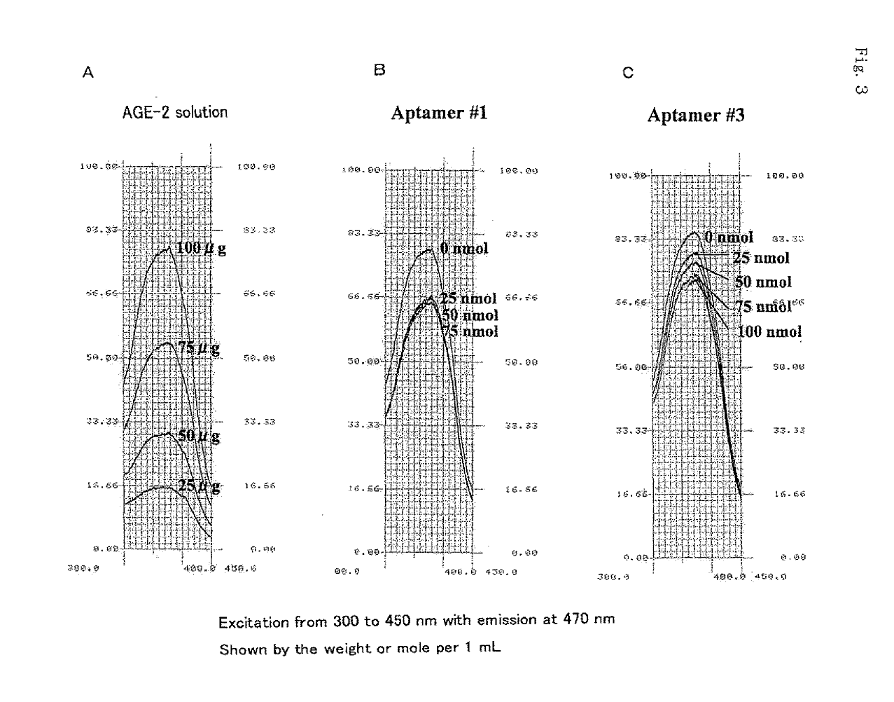

First, for measuring fluorescence properties of AGE-2, wavelengths of AGE-2 were determined for excitation and quenching using a spectrofluorometer (FP-777: JASCO Corporation). As a result, a maximum emission was observed at 380 nm for excitation and at 470 nm for quenching. Subsequently, the fluorescence intensity was measured with 25 to 100 μg/mL of AGE-2 at the excitation wavelength of 380 nm, and a calibration curve was prepared (FIG. 3A).

Then, to each aptamer (SEQ ID NOs: 1 to 15), 100 μg/mL of the AGE-2 was added at a final concentration of 25 to 100 nM, and the fluorescence intensity was measured (see FIGS. 3B and C). Based on attenuation in the fluorescence intensity of AGE-2 when the aptamer was added at 25 nM, the weight (ng) of the bound AGE-2 was calculated per mole (nmol) of the aptamer. The results are shown in Table 3.

TABLE 3

| SEQ ID | Bound AGE-2 per nmol of |
|---|---|
| NO: | Aptamer (ng) |
| 1 | 595.8 |
| 2 | 175.2 |
| 3 | 260.0 |
| 4 | 210.3 |
| 5 | 143.1 |
| 6 | 201.5 |
| 7 | 146.0 |
| 8 | 175.2 |
| 9 | 116.8 |
| 10 | 134.3 |
| 11 | 137.3 |
| 12 | 116.8 |
| 13 | 175.2 |
| 14 | 157.7 |
| 15 | 271.0 |

### Example 4

### Apoptosis Experiment Using Bovine Pericytes

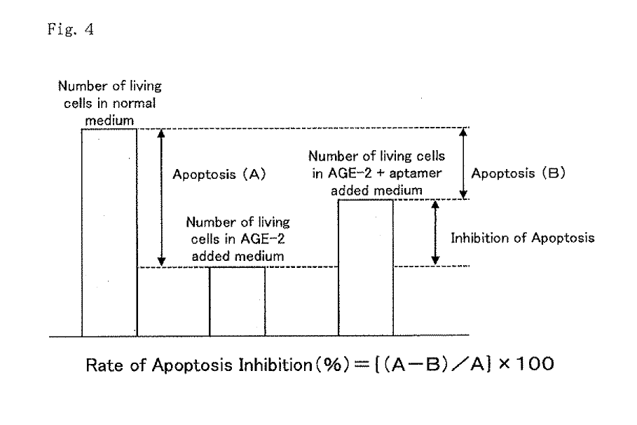

Isolated bovine pericytes from killed bovines were passaged with Dulbecco's Modified Eagle Medium (Gibco BRL, Rockville, Md.) supplemented with 20% fetal bovine serum (ICN Biomedicals Inc., Aurora, Ohio). The resultant bovine pericytes were incubated with 20 μg/mL of AGE-2 and 100 μg/mL of aptamer (SEQ ID NOs: 1 to 22) at 37° C. for two days. Further, incubations were performed as mentioned above, by use of HSA, instead of aptamer, for the control, and with AGE-2 alone for the positive control. After two days, cells were stripped off with trypsinization, and then applied to [³H]-thymidine incorporation, and the number of living cells was counted. Based on the number of living cells, the rate of apoptosis inhibition was calculated as shown in FIG. 4. The results are shown in Table 4.

TABLE 4

| | | Fluorescence | | |
|---|---|---|---|---|
| | | Intensity | | Rate of Apoptosis |
| | | Mean | S.E. | Inhibition (%) |
| Control | | 0.265 | 0.00772 | — |
| AGE-2 alone | | 0.162 | 0.00509 | 100 |
| Aptamer | 1 | 0.222 | 0.0144 | 58.3 |
| | 2 | 0.223 | 0.013 | 59.2 |
| | 3 | 0.186 | 0.00648 | 23.3 |
| | 4 | 0.203 | 0.0111 | 39.8 |
| | 5 | 0.203 | 0.00745 | 39.8 |
| | 6 | 0.203 | 0.0085 | 39.8 |
| | 7 | 0.210 | 0.00712 | 46.6 |
| | 8 | 0.210 | 0.0121 | 46.6 |
| Control | | 0.301 | 0.0124 | — |
| AGE-2 alone | | 0.203 | 0.0128 | 100 |
| Aptamer | 9 | 0.212 | 0.0102 | 9.2 |
| | 10 | 0.223 | 0.0172 | 20.4 |
| | 11 | 0.218 | 0.00765 | 15.3 |
| | 12 | 0.224 | 0.00886 | 21.4 |
| | 13 | 0.268 | 0.017 | 66.3 |
| | 14 | 0.238 | 0.00911 | 35.7 |
| | 15 | 0.244 | 0.0117 | 41.8 |
| Control | | 0.251 | 0.00295 | — |
| AGE-2 alone | | 0.150 | 0.00462 | 100 |
| Aptamer | 16 | 0.189 | 0.00516 | 38.6 |
| | 17 | 0.183 | 0.0047 | 32.7 |
| | 18 | 0.207 | 0.00598 | 56.4 |
| | 89 | 0.210 | 0.00564 | 59.4 |
| | 20 | 0.199 | 0.00334 | 48.5 |
| | 21 | 0.206 | 0.00747 | 55.4 |
| | 22 | 0.221 | 0.0101 | 70.3 |

As shown in Table 4, apoptosis induced by addition of AGE-2 alone was inhibited by adding also the AGE-2 aptamers. Accordingly, it was found that the AGE-2 aptamers could bind to AGE-2 to inhibit functions of AGE-2.

### INDUSTRIAL APPLICABILITY

Since the AGE-2 aptamer of the present invention can be used for detecting AGE-2, it can be used as a reagent for detection/diagnosis of AGE-2 involved diseases such as: diabetic complications such as diabetic retinopathy, diabetic nephropathy, and diabetic neuropathy; neurodegenerative diseases such as Alzheimer's disease; and proliferation, metastasis, and invasion of malignant tumors. Furthermore, since the AGE-2 aptamer of the present invention has an activity for inhibiting AGE-1, it can be used as an agent for preventing/treating AGE-2 involved diseases, such as the diseases mentioned above. In particular, due to increase in diabetic complications in association with increase in diabetes patients, the AGE-2 aptamer of the present invention is useful for early detection and treatment. Moreover, the AGE-2 aptamer of the present invention also can be used for fundamental researches such as elucidation of the onset mechanism of AGE-2 involved diseases.

1. An aptamer that binds to a glyceraldehyde-derived advanced glycation end product (AGE-2), but does not bind to human serum albumin,
wherein the aptamer comprises at least 35 bases, and
the cytosine content in the bases is at least 35%, or the guanine content in the bases is at least 32%.

2. The aptamer of claim 1, wherein the aptamer is a single-stranded DNA.

3. The aptamer of claim 1, wherein the aptamer comprises at least 50 bases and not greater than 120 bases.

4. The aptamer of claim 1, wherein the cytosine content in the bases is at least 40%.

5. The aptamer of claim 4, wherein the cytosine content in the bases is at least 50%.

6. The aptamer of claim 1, wherein the guanine content in the bases is at least 35%.

7. The aptamer of claim 6, wherein the guanine content in the bases is at least 40%.

8. The aptamer of claim 2, wherein the single-stranded DNA comprises a base sequence according to any one of SEQ ID NOs: 1 to 24 in the Sequence Listing.

9. The aptamer of claim 2, wherein the single-stranded DNA comprises a base sequence according to any one of SEQ ID NOs: 25 to 41 in the Sequence Listing.

10. An AGE-2 detection reagent comprising the aptamer of claim 1.

11. An AGE-2 detection kit comprising the AGE-2 detection reagent of claim 10.

12. A diagnostic reagent for an AGE-2 involved disease, comprising the aptamer of claim 1.

13. The reagent of claim 12, wherein the AGE-2 involved disease is a diabetic complication.

14. A diagnostic kit for an AGE-2 involved disease, comprising the reagent of claim 12.

15. The kit of claim 14, wherein the AGE-2 involved disease is a diabetic complication.

16. An anti-AGE-2 agent comprising the aptamer of claim 1.

17. An agent for preenting or treating an AGE-2 involved disease, comprising the aptamer of claim 1.

18. The agent of claim 17, wherein the AGE-2 involved disease is a diabetic complication.
